(19) United States
(12) Patent Application Publication
(10) Pub. No.: US 2005/0085543 A1
(43) Pub. Date: Apr. 21, 2005

(54) Methods of treating bone or cartilage conditions by the administration of creatine

(75) Inventors: Theo Wallimann, Kindhausen (CH); Isabel Gerber, Pieterlen (CH)

Correspondence Address: JONES DAY, 222 EAST 41ST ST, NEW YORK, NY, 10017, US

(73) Assignee: Synthes USA

(21) Appl. No.: 11/005,942

(22) Filed: Dec. 7, 2004

Related U.S. Application Data
(63) Continuation of application No. 09/769,404, filed on Jan. 26, 2001.
(63) Continuation of application No. PCT/EP98/04713, filed on Jul. 28, 1998.

(51) Int. Cl. A61K 31/205; A61K 31/198

(52) U.S. Cl. 514/554; 514/565

(57) ABSTRACT

The method, composition, and use of the composition for healing defects in bone or cartilage tissue in animals and humans caused by trauma or surgery is disclosed. The method includes administration of creatine compounds including analogues or pharmaceutically acceptable salts thereof. Treatment in accordance with the method speeds-up time for and improves the process of healing of defects in bone or cartilage tissue in animals and humans caused by trauma or surgery including acceptance and bonding of artificial implants. The treatment with creatine compounds can be therapeutic for diseased patients, preventive for healthy people, as well as geriatric for elderly people.

### CROSS-REFERENCE TO RELATED APPLICATION

This application is a continuation of International Application No. PCT/EP98/04713, filed Jul. 28, 1998, now pending, the disclosure of which is hereby incorporated herein by express reference thereto.

### FIELD OF INVENTION

This invention concerns the use of creatine compounds including a method for accelerating healing in an animal or human having a defect in bone or cartilage tissue, as well as a composition useful for the treatment of defects in bone or cartilage tissue. The creatine compounds may be incorporated in three dimensional constructs of osteoblasts, chondrocytes, or mesenchymal stem cells designed for tissue engineering of said bone or cartilage defects. Further, the creatine compounds may be used for improving acceptance and osseous integration of bone implants.

### BACKGROUND OF THE INVENTION

Creatine is a compound that naturally occurs in the human body and is found in mammalian brain and other excitable tissues, such as skeletal muscle, heart, and retina. Its phosphorylated form, creatine phosphate, is also found in the same organs and is the product of the creatine kinase reaction utilizing creatine as a substrate. Creatine and creatine phosphate can be synthesized relatively easily and are believed to be non-toxic in mammals.

The use of creatine and analogues thereof for the treatment of diseases of the nervous system has been described in U.S. application Ser. No. 08/336,388, the disclosure of which is hereby incorporated by reference thereto.

Nowhere, however, has the use of creatine kinase or creatine compounds for the treatment of bone and cartilage cells or tissues been specifically disclosed or advocated for the prevention or treatment of bone and cartilage in health and disease.

### SUMMARY OF THE INVENTION

The invention relates to a method of treating at least one bone or cartilage condition which includes administering to an animal a therapeutically effective amount of an agent including creatine, or an analogue or pharmaceutically acceptable salt thereof, to treat bone or cartilage conditions. The animal to be treated may be a mammal, preferably, it may also be a human.

In one embodiment, the bone or cartilage condition includes a bone or cartilage disease, a bone fracture or defect, or a degenerative disease of cartilage. Diseases that can be treated include, but are not limited to, osteoporosis, osteoarthritis, and periodontitis. In another embodiment, the agent is incorporated in a bone or cartilage graft that is applied to the bone fracture or defect. In a preferred embodiment, the agent is incorporated in at least one three dimensional construct of osteoblasts, chondrocytes, or mesenchymal stem cells designed for tissue engineering of the bone or cartilage condition and wherein the construct is administered to the bone or cartilage.

In another embodiment, the method further includes obtaining bone or cartilage forming cells from a healthy individual, culturing the bone or cartilage forming cells in the presence of the agent to form a three-dimensional cell assembly, and transferring the three-dimensional cell assembly to a specific location having a bone or cartilage defect on the patient. In yet another embodiment, the creatine, or analogue or pharmaceutically acceptable salt thereof, includes creatine, creatine phosphate, creatine pyruvate, cyclocreatine, homocreatine, or homocyclocreatine.

In additional embodiments, the agent is administered with at least one of: hormones, including, but not limited to, parathyroid hormone-related protein, thyroid hormone, insulin, a sex steroid, prostaglandins, or glucocorticoids; vitamins, including, but not limited to, 1,25(OH)₂ vitamin D₃ and analogues or metabolites of vitamin D, vitamin C/ascorbate, or retinoids; growth factors, including, but not limited to, insulin-like growth factors (IGF), transforming growth factor b family (TGF-b), bone morphogenic proteins (BMP), basic fibroblastic growth factor (bFGF), platelet derived growth factor (PDGF), or epidermal growth factor (EGF); cytokines, including, but not limited to, interleukins (IL), interferons, or leukaemia inhibitory factor (LIF); matrix proteins, including, but not limited to, collagens, glycoproteins, hyaluronan, or proteoglycans; serum proteins, including, but not limited to, albumin or alpha-2H5 glycoprotein; enzymes, including, but not limited to, metalloproteinases, collagenases, gelatinases, stromelysins, plasminogen activators, cysteine proteinases, or aspartic proteinases; calcium salts; fluoride salts; bone meal; hydorxyapatite; peptides, including, but not limited to, amylin, vasoactive agents, or neuropeptides; antioxidants, including, but not limited to, cysteine, N-acetyl-cysteine, glutathions, or vitamins A, C, D, or E; transferrin; selenium; boron; silicon; or nitric oxide. In a preferred embodiment, the glycoproteins include, but are not limited to, alkaline phosphatase, osteonectin (ON), gamma-carboxy glutamic acid-containing proteins, or arginine-glycine-asparagine-containing proteins. The proteoglycans include, but are not limited to, aggrecan, versican, biglycan, or decorin. In another embodiment, parathyroid hormone is administered intermittently, and is preferably administered with 1,25(OH)₂ vitamin D₃ and analogues or metabolites of vitamin D, calcitonine, estrogen, or bisphosphonates.

In another embodiment, the bone includes cells having osteoblasts, periosteal cell, stromal bone marrow cells, satellite cells of muscle tissue, or mesenchymal stem cells, or a combination thereof.

In still another embodiment, the cartilage including cells having chondroblasts or mesenchymal stem cells. Preferably, the stem cells are cultured as monolayers, micromass cultures, or in a three-dimensional biodegradable scaffold. In another preferred embodiment, the three-dimensional cell assembly has a structure of a seeded sponge, foam, or membrane. In yet another embodiment, 10 to 20 mM of creatine is concentrated in a culture medium containing one of 0.1% to 5% fetal calf serum or 10 to 250 μg of ascorbic acid or an equivalent amount of a pharmaceutically acceptable ascorbate. In another embodiment, the cell culture is started with 2,000 to 100,000 cells.

In yet another embodiment, the agent is essentially free of dihydrotriazine; dicyano-diamide; or creatinine. Preferably, the agent is administered to a human patient in an amount of 1.4 to 285 mg per day.

In another embodiment, the creatine analogue has the general formula:
Z₁-C(-Z₂)-X-A-Y
and pharmaceutically acceptable salts thereof, wherein:
Y is selected from: —CO₂H, —NI—OH, —NO₂, —SO₃H, —C(═O)NHSO₂J, and —P(═O)(OH)(OJ), wherein J is selected from: hydrogen, C₁-C₆ straight chain alkyl, C₃-C₆ branched alkyl, C₂-C₆ straight alkenyl, C₃-C₆ branched alkenyl and aryl;
A is selected from: C, CH, C₁-C₅ alkyl, C₂-C₅ alkenyl, C₂-C₅ alkynyl, and C₁-C₅ alkoyl chain, each having 0-2 substituents which are selected independently from:
   K, where K is selected from: C₁-C₆straight alkyl, C₂-C₆ straight alkenyl, C₁-C₆ straight alkoyl, 3-6 branched alkyl, C₃-C₆ branched alkenyl, C₄-C₆ branched alkoyl, K having 0-2 substituents independently selected from: bromo, chloro, epoxy and acetoxy;
   an aryl group selected from: a 1-2 ring carbocycle and a 1-2 ring heterocycle, wherein the aryl group contains 0-2 substituents independently selected from: —CH₂L and —COCH₂L, wherein L is independently selected from: bromo, chloro, epoxy and acetoxy; and
   —NH-M, wherein M is selected from: hydrogen, C₁-C₄ alkyl, C₂-C₄ alkenyl, C₁-C₄ alkoyl, C₃-C₄ branched alkyl, C₃-C₄ branched alkenyl, and C₄-C₆ branched alkoyl;
X is selected from: NR₁, CHR₁, CR₁, O and 5,
   wherein R₁ is selected from:
      hydrogen,
      K where K is defined above; and
      an aryl group selected from: a 1-2 ring carbocycle and a 1-2 ring heterocycle, wherein the aryl group contains 0-2 substituents independently selected from: —CH₂L and —COCH₂L where L is defined above;
   a C₅-C₉ Alpha-amino-omega-methyl-omega-adenosyl carboxylic acid attached via the omega-methyl carbon;
   a C₅-C₉ Alpha-amino-omega-aza-omega-methyl-omega-adenosylcarboxylic acid attached via the omega-methyl carbon; and
   a C₅-C₉ Alpha-amino-omega-thia-omega-methyl-omegaadenosylcarboxylic acid wherein A and X are connected by a single or double bond;
Z₁ and Z₂ are chosen independently from: ═O, —NHR₂, —CH₂R₂, —NR₂OH; wherein, Z₁ and Z₂ may not both be ═O and wherein R₂ is selected from:
   hydrogen;
   K, where K is defined above;
   an aryl group selected from: a 1-2 ring carbocycle and a 1-2 ring heterocycle, wherein the aryl group contains 0-2 substituents independently selected from: —CH₂L and —COCH₂L where L is as defined above;
   a C₄-C₈ Alpha-amino-carboxylic acid attached via the omega-carbon;
   B, wherein B is selected from: —CO₂H, —NHOH, NO₂, —SO₃H, —C(═O)NHSO₂J and —P(═O)(OH)(OJ), wherein J is as defined above:
   D-E, wherein D is selected from: C₁-C₃ straight chain alkyl, C₃ branched alkyl, C₂-C₃ straight alkenyl, C₃ branched alkenyl, C₁-C₃ straight alkoyl, and aryl; and E is selected from: —(PO₃)ₙNMP, where n is 0-2 and NMP is a ribonucleotide monophosphate connected via the 5′-phosphate, 3′-phosphate or the aromatic ring of the base; —[P(=0) (OCH₃)(O)]ₘ-Q, wherein m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose of the aromatic ring of the base; and an aryl group containing 0-3 substituents chosen independently from: Cl, Br, epoxy, acetoxy, —OG, —C(═O)G, and —CO₂G, where G is independently selected from: C₁-C₆ straight alkyl, C₂-C₆ straight alkenyl, C₁-C₆ straight alkoyl, C₃-C₆ branched alkyl, C₁-C₆ branched alkenyl, C₄-C₆ branched alkoyl wherein E may be attached at any point to D, and if D is alkyl or alkenyl, D may be connected at either or both ends by an amide linkage; and
   E, wherein E is as defined above, provided that:
when E is aryl, B may be connected by an amide linkage;
if R₁ and at least one R₂ group are present, R₁ may be connected by a single or double bond to an R₂ group to form a cycle of 5 to 7 members;
if two R₂ groups are present, they may be connected by a single or double bond to form a cycle of 5 to 7 members; and
if R₁ is present and or Z₂ is selected from —NHR₂, —CH₂R₂ and —NR₂OH, then R₁ may be connected by a single or double bond to the carbon or nitrogen of either Z₁ or to form a cycle of 4 to 7 members.

The invention also relates to a method of promoting growth and mineralization of bone or cartilage cells and tissues that includes administering to a subject in need of such treatment a therapeutically effective amount of an agent including creatine, or an analogue or pharmaceutically acceptable salt thereof, to promote growth and mineralization of bone or cartilage therein.

The invention further relates to a method of improving acceptance and osseous integration of bone implants that includes administering to a subject in need of such treatment a therapeutically effective amount of an agent including creatine, or an analogue or pharmaceutically acceptable salt thereof, to improve acceptance and osseous integration of bone implants.

The invention also relates to a method for accelerating healing in a subject having a defect in bone or cartilage tissue caused by trauma, surgery, or a degenerative disease, including administering to the subject a therapeutically effective amount of a creatine compound, analogue, or pharmaceutically acceptable salt thereof, or a creatine kinase.

The invention relates to a composition useful for the treatment of defects in bone or cartilage tissue of animals or humans caused by trauma or surgery, including a creatine compound, analogue, or pharmaceutically acceptable salt thereof, the composition being suitable for oral administration and including a pharmacologically suitable carrier to improve bioavailability. Preferably, the carrier is carbohydrates, maltodextrins, or dextrose.

The invention further relates to a method of preparing an agent for treatment of bone or cartilage cells or tissues, including removing bone or cartilage forming cells from a healthy subject, adding the bone or cartilage forming cells to a cell structure, transfecting the bone or cartilage forming cells with complimentary DNA coding for creatine kinase isoforms and made to overexpress creatine kinase isoenzyme(s), and expanding and cultivating the bone or cartilage forming cells to form in vitro genetically engineered cartilage or bone tissues transplantable into areas of cartilage or bone defects of the healthy subject.

### BRIEF DESCRIPTION OF THE DRAWINGS

Further features and advantages of the invention can be ascertained from the following detailed description provided in connection with the drawing(s) described below:

FIG. 1 is a graph showing Viability (NR) of monolayer osteoblast cell cultures at 1, 2, and 3 weeks in the absence (control) and presence of either 10 mM or 20 mM creatine in the medium;

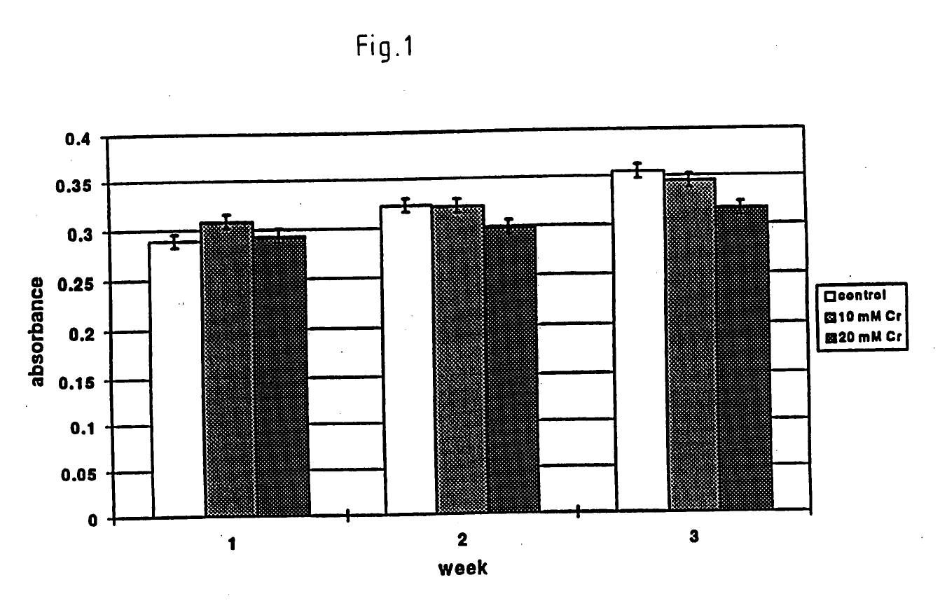

FIG. 2 is a graph showing metabolic activity (MTT) of monolayer osteoblast cell cultures at 1, 2, and 3 weeks in the absence (control) and presence of either 10 mM or 20 mM creatine in the medium;

FIG. 3 is a graph showing mineralization of monolayer osteoblast cell culture at 2 and 3 weeks in the absence (control) and presence of either 10 mM or 20 mM creatine in the medium;

FIG. 4 is a graph showing mineralization of micromass osteoblast cell culture at 2 and 3 weeks in the absence (control) and presence of either 10 mM or 20 mM creatine in the medium; and

FIG. 5 is a graph showing embryonic rat femora wet weight after 3 weeks in organ culture, with and without 10 mM or 20 mM creative.

### DETAILED DESCRIPTION OF THE PREFERRED EMBODIMENTS

The present invention provides for use of creatine kinase and creatine compounds, which modulate one or more of the structural or functional components of the creatine kinase/creatine phosphate system, as therapeutic agents. More particularly, the present invention provides methods of one or more of the following:
a) treatment of bone or cartilage diseases (e.g., osteoporosis, osteoarthritis or periodontitis);
b) promoting growth or mineralization of bone or cartilage cells and tissues;
c) conservative or operative treatments of bone fractures or bone defects;
d) applying bone or cartilage grafts to bone or cartilage fractures or defects;
e) tissue engineering by extracorporeal culture of bone or cartilage forming cells (obtained from a healthy individual or particular patient) in the presence of creatine to form a three-dimensional cell assembly which can be transferred in a subsequent step to a specific location having a bone or cartilage defect of the same particular patient; and
f) metabolic engineering of bone and cartilage cells by transfection with DNA coding for creatine kinase in order to make said cells overexpress creatine kinase and thus, together with creatine, improve, stimulate, and stabilize the physiological function of said cells and tissues for reimplantation into patients as outlined in section e.

In all of these applications of creatine according to the invention, the essential function of creatine is its ability to act as an energy source and regulator of cellular energy metabolism, as well as a cell protective agent against metabolic stress. In addition, creatine has been surprisingly shown to exert a protective effect on early events of programmed cell death or apoptosis. These effects are all mediated by creatine kinase.

The surprising effect of the creatine compounds on bone and cartilage cells and tissues has been to reduce time for and improve the process of healing wounds in bone or cartilage tissue caused by trauma or surgery, including bone fractures and the acceptance and bonding of artificial implants. The treatment with creatine compounds can be therapeutic for diseased patients, preventive for healthy people, as well as geriatric for elderly people. A variety of creatine compounds may be used in connection with the invention, in particular including creatine, creatine phosphate, creatine pyruvate, and cyclocreatine.

The creatine compounds may be in the form of a pharmacologically acceptable salt, or combined with an adjuvant or other pharmaceutical agent effective to treat bone or cartilage cells. Compounds useful in the present invention are creatine compounds, which modulate the creatine kinase system.

The present invention also provides pharmaceutical compositions containing creatine compounds in combination with a pharmaceutically acceptable carrier. Suitable carriers are disclosed in “Principles of Tissue Engineering”, Chapter 19: Biodegradable Polymers for Tissue Engineering, J. M. Pachence and J. Kohn, 1997, pp. 274-293; and “Der orthopade, Bone replacement materials”, J. M. Rueger, 2-1998, pp. 73-79, the disclosures of which are hereby incorporated by reference thereto.

The compositions of the invention may be administered orally, in the form of granulates, or in a sustained-release formulation. “Sustained release” means a formulation in which the composition becomes biologically available to the patient at a measured rate over a prolonged period. Such compositions are well known in the art.

The main route of creatine biosynthesis in mammals involves the formation of guanidinoacetate in the kidneys, its transport through the blood, and its methylation to creatine in the liver. Creatine, exported from the liver and transported again through the blood, may then be taken up by the creatine-requiring tissues via the creatine transporter protein. When mammalian cells are cultured, creatine is available only in the amounts present in the serum added, which contains 0.05 to 0.10 mM Creatine.

The term “mammals” is used in its conventional sense to include animals and especially humans, with the terms “subject” or “patient” being used to refer generically to any of these mammals.

The enzyme creatine kinase (CK) plays a key role in the energy metabolism of cells that have intermittently high and fluctuating energy requirements. CK isoenzymes are found predominantly in skeletal and cardiac muscle, but also in spermatozoa (vertebrate and sea urchin sperm), electrocytes of the electric organ of electric fish, photoreceptor cells of the retina and the lens of the eye, brain (glial and neuronal cells of the cerebellum, glomerular structures of the cerebellum, neurones), the uterus and placenta, intestinal brush border epithelial cells and endothelial cells, kidney and rectal salt glands, adipose tissue, pancreas, thymus, thyroid and liver, cartilage and bone, macrophages, blood platelets, as well as in certain malignant tumors and cancer cells.

The reaction catalyzed by CKs, the reversible transfer of the phosphoryl group from phosphocreatine (PCr) to ADP, allows regeneration of the key cellular energy carrying molecule ATP. Cells contain a number of different CK isoforms, which are not evenly distributed in cells. They are compartmentalized in an isoform-specific fashion the two isoforms M-CK and B-CK are cytosolic, and two of the isoforms Mia-CK and Mib-CK are specifically mitochondrial. These various isoforms of CK are believed to constitute an intricate energy buffering and transport system, connecting sites of high energy phosphate production (by glycolysis and oxidative phosphorylation) to sites of energy consumption (ATPases).

The mitochondrial CK isoforms (Mi-CK) are located along the outer surface of the entire inner membrane, and also at sites where the inner and outer membranes are in close proximity. At these latter sites, Mi-CK can directly use intra-mitochondrially-produced ATP to generate PCr, which is exported to the cytosol where it serves as an easily diffusible, energy-storage metabolite. In contrast to the cytosolic CK isoforms, which are dimeric, Mi-CK, forms highly symmetrical, cube-like octamers that can bind to the periphery of lipid membranes. Most importantly, Mi-CK can mediate contact-site formation between the inner and the outer mitochondrial membranes and, in addition, Mi-CK is functionally coupled to oxidative phosphorylation by the adenine nucleotide transporter that catalyzes ATP/ADP antiport across the inner membrane. Net PCr production can be stimulated by the addition of extra-mitochondrial Creatine, even in the presence of external ATP-regeneration systems and ATP sinks.

Creatine and Phosphocreatine in Cartilage

Resting and hypertrophied cartilage both contain PCr. The distribution of PCr, however, varies in the different zones of the cartilage. The highest content of creatine is in the resting cartilage. The other zones have similar amounts of creatine. On the other hand, the highest amount of PCr is found in the proliferative zone of cartilage with lower concentration in resting and hypertrophic cartilage. In calcified cartilage-bone, PCr is not detectable.

Experimental studies have shown that external addition of PCr promotes cartilage mineralization in organ and cell cultures. The deposition of calcium in the cartilage matrix of the epiphysis of cultured embryonic chick femora is accelerated by the addition of very crude preparations of PCr and creatine at 0.1 mM in chick embryo extract with 20% horse serum. Mineralization in differentiating chick limb bud mesenchymal cells in micromass cultures is promoted by the addition of 1 and 2 mM ATP or 2 mM PCr. The formed mineralized cartilage matrix is similar to that in ovo. The addition of ATP or PCr does not alter the rate of cell proliferation, the rate of matrix synthesis, the mean crystallite length, or the rate of mineral deposition, when contrasted with cultures supplemented with inorganic phosphate. The ultrastructure of the cultured cells in the presence of 4 mM inorganic phosphate (Pi), 1 to 2 mM ATP or 2 mM PCr are similar at days 14 and 21. There are differentiated chondrocytes within the nodule containing hypertrophied and degenerating cartilage. At the edge of the nodule, the cartilaginous, matrix containing type II collagen, proteoglycans and matrix vesicles is surrounded by undifferentiated cells and type I collagen. ATP, PCr, or Pi increase the mineral to matrix ratio around the edge of the micromass, but not in the center of the cartilage nodule (low mineral to matrix ratio). There is no difference in the pattern of mineralization due to Pi, ATP, or PCr.

Reduction of the creatine uptake by feeding rats with beta-guanidinopropionate (GPA) results in marked abnormalities in the epiphyseal growth plate of the rats. The zone of calcified cartilage is wider and extends into the metaphysis. The hypertrophic chondrocytes are vacuolated and poorly columnated, and mineralization is less abundant and also occurs in the transverse septa Vascular invasion is poor. There is a reduction in the osteoid formation. GPA interferes with the synthesis of pro-a type II and type X collagen in cultured chondrocytes.

Creatine and Phosphocreatifle in Bone

PCr increases the alkaline phosphatase (ALP) activity in SaOS-2 cells. The perichondral ossification in the diaphysis of cultured embryonic chick femora is accelerated by the addition of PCr and Creatine preparations at 0.1 mM to chick embryo extract with 20% horse serum.

Creatine Kinase in Cartilage

The level of CK activity is correlated to the chondrocyte maturation in the epiphysis and in the rib. There is a six-fold increase in CK activity from the resting-proliferative cartilage to the hypertrophic cartilage and a seventeen-fold increase in the calcified cartilage-bone zone. In resting and proliferating cartilage, the predominant CK isoform is MM. M-CK is ⅓ to ⅕ of those in skeletal muscles (160,000 ng/mg protein), and the amount is independent of the age. In hypertrophic cartilage, the MB-CK and BB-CK isoforms are predominant and B-CK is 30 to 47-fold higher than in skeletal muscle (60 ng/mg protein and B-CK shows a significant decrease with advancing age.

CK activity seems to be required for matrix synthesis, and mineralization of the enchondral growth cartilage and chondrocytes in culture undergoing hypertrophy show an increase in the CK activity. CK activity peaks in the cartilage in rats of peripubertal age.

CK activity in the cartilage is stimulated by growth hormone (GH), by insuline-like growth factor 1 (IGF-I), by a metabolite of vitamin D [24R,25(OH)₂D₃] in normal rats and in vitamin D-deficient rats, by PTH, by protease-resistant variants of parathyroid hormone (PTH), and by 17b-estradiol in normal rats and in ovariectomized rats. Stimulation of BB-CK activity is followed by a parallel increase in DNA synthesis. In rachitic cartilage, the profile of CK is similar, but the values in the hypertrophic and also in the calcified cartilage are lower than in the normal cartilage.

Creatine Kinase in Bone

In embryonic chick bone, there is BB-CK along with some MB and MM-CK activity. During early facial development, there is a prominent anaerobic metabolism in the facial processes, BB-CK is present from the 9th embryonic day, and during the 10th to 15th days, MB-CK and MM-CK develop. The amount of bone produced during hetereotropic bone formation by implantation of BMP into muscles of rats shows an almost parallel relationship with the levels of S-100b protein, B-CK, hyaluronic acid, and chondroitin sulphate and the activity of ALP. B-CK expression is modulated by transcriptional and posttranscriptional mechanisms during differentiation of osteoblastic cells. Enhanced activity of membrane pumps and changes in the cytoskeleton are related to the formation of extracellular matrix and mineralization.

In bone, similar to cartilage, BB-CK is also experimentally increased both in vitro and in vivo by IGF-I by 1,25(OH)₂D₃ by PTH by protease-resistant variants of PTH and by PGE₂ by 17b-estradiol (E2). Furthermore, the stimulation of the bone-cell energy metabolism by 17b-estradiol (E₂) and testosterone is sex specific, as shown in diaphyseal bone of weanling rats, but not in epiphyseal cartilage. E₂ causes a 70 to 200% increase in CK activity in vivo and in vitro in ROS 17/2.8, in MC3T3-E1 cells and foetal rat calvaria cells, and a 40% increase in rat epiphyseal cartilage cells. The stimulation of E₂ is dose- and time-dependent. Ovariectomized rats 1 to 4 weeks after surgery show a stimulation of CK by E₂, 24 hours after injection. Both the basal and stimulated activity of CK is higher in the diaphysis and epiphysis than in the uterus. The effect of E₂ in vivo and in chondroblasts and osteoblasts in vitro is inhibited by high levels of the anti-oestrogen tamoxifen which by itself, in high concentrations, shows stimulatory effects. Furthermore, gonadectomy causes a loss of the sex-specific response of diaphyseal bone to steroid hormones. CK activity peaks in diaphyseal bone and cartilage in rats of peripubertal age. Patients with autosomal-dominant osteopetrosis Type II have an elevated level of BB-CK, but patients with other sclerosing bone diseases do not show such an elevation in BB-CK.

For adult humans (70 kg) the daily dosage of chemically pure creatine monohydrate is typically in the range of 0.1 to 20.0 grams per day, preferably with a loading phase of 4 times 4 to 6 grams per day for the first 8 to 14 days, and a maintenance dosage of 2 to 4 grams per day for another 3 months, with an interruption of the supplementation scheme for one month thereafter. To improve bioavailability, chemically pure creatine monohydrate can be mixed with carbohydrates like maltodextrins, dextrose, and others.

The various features of novelty that characterize the invention are pointed out with particularity in the claims annexed to and forming part of this disclosure. For a better understanding of the invention, its operating advantages, and specific objects attained by its use, reference should be had to the accompanying drawings, examples, and descriptive matter in which are illustrated and described preferred embodiments of the invention.

The effects of supplementation with creatine and beta-guanidinopropionic acid (GPA; a creatine analogue and competitor of creatine uptake into the cell) on the differentiation of osteoblasts and chondrocytes in vitro were determined. The parameters investigated were viability (based on the physical uptake of neutral red and the metabolic activity), histochemical ALP activity and degree of mineralization, as well as the TEM ultrastructure.

Cell Culture

This isolation technique is based on the ability of osteoblasts to migrate from bone onto a substratum. Parietal and frontal calvariae (4 per animal) were aseptically explanted from 6 day-old IcoIbm rats. The calvariae were placed in Tyrode's balanced salt solution, calcium and magnesium free (TESS). The periosteum was enzymatically removed with 0.05% trypsin and 0.02% collagenase A (0.76 U/mg) dissolved in TBSS (40 calvaria/20 ml). The calvariae were shaken for 70 minutes in a waterbath at 37° C. They were washed with TESS and then transferred to 60 mm culture dishes (40 calvariae/dish) containing 5 ml of 0.02% collagenase A (0.76 U/mg) in culture medium BGJ_{b} Fitton-Jackson modification and placed in the incubator for 4 hours. The calvariae were then washed with culture medium B supplemented with 10% foetal calf serum (FCS). The calvariae were transferred into 60 mm culture dishes (4 frontal and 4 parietal/dish). The growth medium supplemented with 10% FCS and 50 μg/ml ascorbate was completely changed every 48 hours. The culture was kept for 3 weeks.

After 3 weeks the migrated cells were harvested. The dish was washed with TESS, and 5 ml of TESS containing 0.05% trypsin and 0.02% collagenase A (0.76 U/mg) was added. After 1 hour in the incubator, the dish was washed with culture medium BGJ_{b} supplemented with 10% FCS. The dishes containing the calvariae and cells were rinsed with serum containing media BGJ_{b}. The cells obtained were filtered through a 40 μm nylon mesh to remove bone debris and cell aggregates. The suspended cells were centrifuged at 600 g for 5 minutes. The cell pellet was resuspended in serum containing medium BGJ_{b} and centrifuged. The viability of the resuspended cells was examined by the dye exclusion of 0.4% trypan blue, and the cells were counted in a haeinocytometer. The inoculation densities were 2·10⁵/10 cm² for monolayer and 2·10⁵/30 μl for micromasses. The micromass cultures were kept for 1 hour in the incubator before 2 ml growth medium was added.

Organ Cultures

Calvariae With Periosteum

Parietal and frontal calvariae (4 per animal) were aseptically explanted from 6 day-old IcoIbm rats. The calvariae were washed thoroughly with TBSS, then transferred into 60 mm culture dishes (4 frontal and 4 parietal/dish) containing growth medium BGJb supplemented with 50 μg/ml ascorbate either serum-free or with 10% FCS. The medium was changed completely every 48 hours. The culture was kept for 3 weeks and then processed for histology.

Denuded Calvariae

The periosteuin was enzymatically removed with 0.05% trypsin and 0.02% collagenase A (0.76 U/mg) dissolved in TBSS (40 calvariae/20 ml). The calvariae were shaken for 70 minutes in a water bath at 37° C. They were washed with TBSS. The calvariae were then transferred to 60 mm culture dishes (40 calvariae/dish) containing 5 ml of 0.02% collagenase A (0.76 U/mg) in culture medium BGJ_{b} and placed in the incubator for 4 hours. The calvariae were then washed with culture medium BGJ_{b} supplemented with 10% ECS. The calvariae were transferred into 60 mm culture dishes (4 frontal and 4 parietal/dish). The growth medium BGJ_{b}, supplemented with 50 μg/ml ascorbate, was either used serum-free or with 10% FCS, and was completely changed every 48 hours. To study the effect of FCS, the cultures were kept for 3 weeks and then processed for histology.

To study the bone regeneration capacity of calvariae, they were kept as long-term cultures for 6, 9, 12, 15 weeks in growth medium with 10% FCS. Every 3 weeks, these calvariae were transferred into a fresh culture dish. At the endpoint, the calvariae were processed for histology.

Embryonic Long Bones

The rats were sacrificed on the 17^{th} to 18^{th} day of pregnancy. The embryos were aseptically removed from the uterus, and both femora were carefully dissected free into sterile TBSS under the stereo-microscope. Organ-culture of the rudiments was performed in 10 cm² plastic culture dishes. A Teflon carrier with a nylon mesh (20 μm pore size) was mounted in the dish, keeping the explants floating and ensuring optimal gas exchange and nutritional conditions. The right and the left femora from each animal were randomly assigned to the experimental or control group. The control groups were kept in 3 ml B with 50 μg/ml ascorbate. In the experimental group, the growth medium was supplemented with either 10 mM creatine, 20 mM creatine, 1 mM GPA, 5 mM GPA, or 10 mM GPA. The growth medium was renewed every second day until day 10. Culture was carried out at 37.5° C. and in a 5% CO₂ atmosphere. At 10 days, the wet weight of each femora was determined on a microbalance. The result of each experimental femora was expressed relative to its collateral control. For the histological evaluation, the femora as fixed in 4% formaldehyde, dehydrated, and embedded in methylmethacrylate. The 6 μm sections were stained by Pentachrome-Movat.

Culture Condition

All the cultures were kept at 37° C. in a humidified atmosphere of 5% CO₂, 95% air. All culture media were supplemented with 50 μg/ml ascorbate. To analyze the collagen types, 60 μg/ml beta-aminopropionitrile (beta-APN) was added to the culture medium. During cell isolation and inoculation, no ascorbate was used to increase plating efficiency. No antibiotics and no beta-glycerophosphate were added. The media were completely changed every 48 hours (60 mm culture dish 5 ml; 35 mm culture dish 2 ml).

Alkaline phosphatase activity (histochemically)

The cells were histochemically stained for the alkaline phosphatase as described in the Sigma Technical Bulletin No. 85L.

Gently fixed cells were incubated in a solution containing naphtol AS-MX. As a result of phosphatase activity, naphtol AS-MX was liberated and immediately coupled with a diazonium salt forming an insoluble, blue pigment at sites of phosphatase activity.

Solutions

Fixative

2 vol. Citrate buffer; dilute citrate concentrate 1:50

3 vol. acetone

Stain

dissolve content of 1 capsule Fast Blue in 48 ml distilled water on a magnetic stirrer.

add 2 ml of naphtol AS-MX solution just before use.

Procedure

1. wash 3 times in TBSS

2. fixation 5 mm. at 20° C.

3. wash 3 times with distilled water

4. stain 30′ in the dark at room temperature (RT)

5. wash 3 times with distilled water.

Mineralization

The most specific method for detecting calcified matrices is the von Kossa reaction. Silver staining indicates the presence of calcium phosphate aggregated with certain organic acids. Structural details are completely obscured by the dark precipitate. Calcified tissue components are darkened in various shades from light brown to deep black, irrespective of their mineral content.

Solution

Silver nitrate

5% AgNO₃ in distilled water

Pyrogallol

1% in distilled water

Sodium thiosulphate

1% Na₂S₂O₃5 H₂0 in distilled water

Procedure

| | | |
|---|---|---|
| 1. | fixation in 4% formaldehyde | 30 min. |
| 2. | wash in distilled water | 3 times |
| 3. | silver nitrate | 30 min. in the dark |
| 4. | wash with distilled water | 5 min. in the dark |
| 5. | pyrogallol | 5 min. |
| 6. | wash with distilled water | 5 min. |
| 7. | sodium thiosulphate | 10 min. |
| 8. | wash with distilled water | 5 min. |

TEM preparation

In an electron microscope, the specimen is exposed to very high vacuum. Therefore, the tissue has to be fixed and stained with heavy metals to give contrast and only very dense material deflects electrons and forms images. The tissue is impregnated with heavy metals (e.g., uranium, lead) before or after sectioning. Because electrons do not penetrate very deeply into the tissue, very thin sections (50 to 100 nm) have to be cut with either a glass or a diamond knife on an ultra microtome. For ultrathin sectioning, the specimen has to be dehydrated and penetrated with monomeric resin which polymerizes.

For chemical fixation, glutaraldehyde is mostly used. Glutaraldehyde cross-links the proteins covalently to their neighbors. In order to stabilize the lipids, especially the cell membranes, osmiumtetroxide is used as a postfixation. To enhance the contrast, the tissue is treated en block with uranyl acetate and the sections are subsequently stained with uranylacetate and lead citrate.

Solutions

0.2 M Cacodylate buffer pH 7.4

Stock A 25 ml

Stock B 1.35 ml

distilled water ad 100 ml

Stock A 10.7 g Cacodylic acid sodium salt Trihydrate

250 ml Distilled water

Stock B 0.2 M HCl

Fixation

25% glutaraldehyde (EM grade) 2 ml

0.2 M cacodylate buffer pH 7.4 10 ml

distilled water ad 20 ml

Postfixation 1% 0s0₄ in 0.1 M cacodylate buffer pH 7.4 1 vol. 2% 0604

1 vol. 0.2 M Cacodylate buffer pH 7.4

2% OSO₄

fracture glass vial

add distilled water

sonicate 5 min.

filter through 0.45 mm filter (Millex)

keep in dark at 4° C.

2% aqueous uranyl acetate

Procedure

| | | |
|---|---|---|
| 1. | Fixation at 20° C. | 20 min. |
| 2. | rinse in 0.1 M cacodylate buffer pH 7.4 | 3 times 30 seconds |
| 3. | post-fixation at 4° C. | 1 hour |
| 4. | rinse in distilled water | 3 times 30 seconds |
| 5. | uranyl acetate at room temperature. | 1 hour |
| 6. | dehydration in a graded series of ethanol: | |
| 7. | 70%, 80%, 90%, 100%, 100%, 100%. | every 5 min. |
| 8. | LR White (Polysciences). | >2 hours |
| 9. | Polymerization at 60 W. | overnight |

Ultrathin sections were cut either with a glass knife or with a Drukker Diamond knife on a LKB III Microtome, placed on Formvar coated copper grids, and stained with heavy metals.

Solutions

5% uranyl acetate

1 g/20 ml

lead citrate according to Reynolds

| | |
|---|---|
| Pb(N0₃)₂ | 0.67 g |
| Sodium citrate | 0.88 g tri sodium citrate dihydrate |
| | 15 ml distilled water |

gentle shaking for 15 minutes.

add 4 ml 1 N NaOH, white precipitate dissolves

fill up to 25 ml distilled water

add distilled water to 25 ml

Filter both solutions through a Whatman No. 50 (hardened) before use.

Procedure

All solutions were placed as drops on a parafilm. Individual grids were placed onto the droplets, to floating, section side down. Solid NaOH pellets were placed in a plastic dish in the same chamber to absorb CO₂ from the air to prevent carbon dioxide precipitation of lead salts. Both the staining solutions and the solid NaOH pellets were covered with a lid.

| | | |
|---|---|---|
| 1. | distilled water | |
| 2. | 5% uranyl acetate | 10 mm. |
| 3. | distilled water | 2 times |
| 4. | lead citrate | 10 mm. |
| 5. | M NaOH | 3 times 30 seconds |
| 6. | distilled water | 2 times |

| | |
|---|---|
| 7. | remove the remaining small amounts |
| | of water between the prongs of the |
| | forceps with filter paper and dry the grids |
| | on Whatman No. 50 filter paper with the |
| | section side up. When the grids were dry, they |
| | were placed in the storage box ready for use. |

The sections were examined on a JEOL JEM 100 CX transmission electron microscope operated at 100 KV. Micrographs were taken on Kodak EM 4303 film at standard magnifications of 2000, 5000, 20000, or 33000 times. Pictures were printed onto multigrade paper.

Cell Viability (MTT)

The Böhringer Cell Proliferation Kit I (MTT) was used for the assay, but we used a different solvent to dissolve the MTT crystals.

Originally, Mosmann, 1983 described the general principle involved in the detection of cell growth/cell death as indicated by the conversion of the tetrazolium salt (MTT) to the colored formazan by mitochondrial dehydrogenases. The concentration of this can then be measured spectrophotoinetrically.

Procedure

1. MTT Stock (5 mg/ml in sterile PBS) from Böhringer was diluted 110 with complete growth medium and sterile filtering.

2. The cells were incubated in 2 ml/10 cm² MTT solution at 37° C. for 3 hours.

3. The supernatant was carefully removed.

4. 4 ml/cm²dimethylsulphoxide (DMSO) was added.

5. The dishes were placed on a shaker until the crystals were completely dissolved.

6. The absorbance of the supernatant (3 aliquots/dish) was read at 550 nm versus DMSO.

If the absorbance was higher than 1, the samples were diluted with DMSO.

Cell Viability (Neutral Red, NR)

The method described in (Lindl et al. 1994) was used.

The uptake of NR into lysosomes is independent of the metabolic status of the cell.

Solutions

0.5 mg Neutral red/ml growth medium, warmed up to 37 ° C. for at least 2 hours,

sterile filtering

Extraction buffer

50% ethanol in 1% acetic acid

Procedure

1. The cells were incubated in 2 ml/10 cm²NR solution at 37° C. for 3 hours.

2. The supernatant was removed,

3. washed with PBS, at least 3 times, until no crystals were present.

4. Addition of 4 ml/10 cm² extraction buffer.

5. The absorbance of the supernatant (3 aliquots/dish) was read at 540 nm versus extraction buffer.

6. If the absorbance was higher than 1, the samples were diluted with extraction buffer.

The mean value and the standard deviation consisted of n independent experiments. The values for the individual experiments were gained from the mean of 3 aliquots of the same dish. To compare the treatment, contrasts analysis of variance models were evaluated.

In experiments carried out as paired designs, a model accounting for the animals considered as blocks was examined. Main effects and interaction effects were examined by F-Tests.

Least Squares Means were calculated to yield average means accounted for the other variables in the model. LS Means were compared by using Tukey's multiple range test.

QQ-Plots of the residuals and Tukey-Anscombe plots (residuals x predicted) were analyzed to check for normal distribution assumption.

Monolayer Cell Culture

Cell Viability and Metabolic Activity

With respect to cell viability, in all groups, neutral red (NR) stained mainly the cells at the edge and the top of the nodules, as well as the cells between them. Staining with trypan blue showed that the cells/matrix between the nodules and the nodules themselves were stained.

Preliminary quantitative data on the NR uptake showed that the Creatine and the GPA groups had similar results as the control group at 2 weeks. Concerning the metabolic activity measured by the MTT reaction, the creatine groups were slightly stimulated when compared to the control, but the 5 M or the 10 mM GPA had lower values than the control, indicating some inhibition of the GPA at these particular concentrations. The 1 mM GPA group was similar to the control. At 3 weeks, all experimental groups had a lower NR uptake than the control. The creatine stimulated the MTT reaction, and the 1 mM or 5 mM GPA had lower values than the control. The 10 mM GPA was comparable to the control. These results indicated that the creatine had a stimulatory effect on the metabolism of the cells and the GPA had some inhibition on the mitochondrial activity of the cells.

In the further experiments to quantify the viability and the metabolic activity of the cells, only the creatine groups were used. Statistical analysis of the NR uptake (IG. 1) showed that there was a small but significant interaction effect (p<0.05). This meant that the effect of treatment with creatine was not similar at the different time points. The NR uptake of the control group at 1 week was significantly lower than that of 2 and 3 week (p<0.03, respectively p<0.0002). The NR uptake of the 10 mM creatine group was significantly higher at 3 weeks as compared to that at 1 week (p<0.02). At 1 and 2 weeks, there was no significant difference between the groups. At 3 weeks, the control group was significantly (p<0.008) higher than the 20 mM creatine group. The increase in the NR uptake of the control group during the culture indicated that there was an increase in the cell number. The difference of the control group and the 10 mM creatine was not significant. This showed that there was no toxic effect of the creatine at this particular concentration. This was in contrast to the 20 mM creatine, which had an significantly lowered NR uptake compared to the control group. This indicated some toxic effects on the proliferation of the cells.

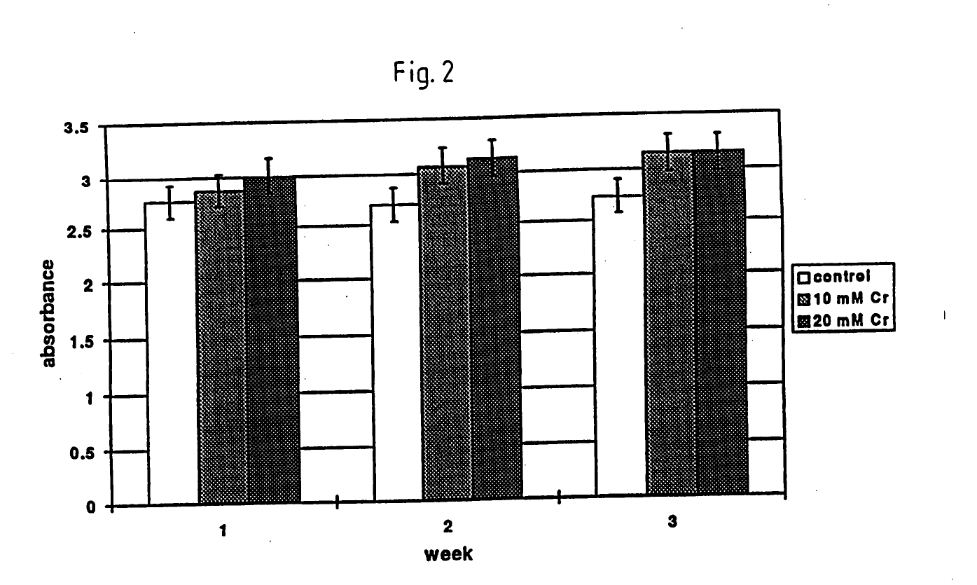

Concerning the metabolic activity (MTT) of the cells (FIG. 2), creatine had an effect on osteoblasts in culture. At 1 week, all groups were similar. At 2 weeks, the control group was significantly lower than the 10 mM creatine and the 20 mM creatine (p<0.015, respectively p<0.0025). At 3 weeks, both the 10 mM creatine and 20 mM creatine were significantly higher than the control group (p<0.001). These data showed that, in general, creatine stimulated the metabolic activity of osteoblasts from the second week on.

Morphology

After 1 week, the cells in all groups formed a monolayer with ALP positive cells. Some cells had a really high ALP activity. After 2 weeks, all groups formed some small mineralized nodules. After 3 weeks, the overall staining for ALP activity was similar in all groups. At higher magnification, the GPA groups showed a different staining pattern for the ALP activity compared with the control and the creatine groups. The cell density around the nodules was lower than in the control and the creatine groups. At 3 weeks, the mineralized nodules increased in size and number compared with 2 weeks. All the experimental groups showed a higher mineralization than the control group. The calcification pattern of the GPA groups was different from the control and the creatine groups, in such that the mineralization was not limited to the nodules and more single cells showed calcification than the control and creatine groups.

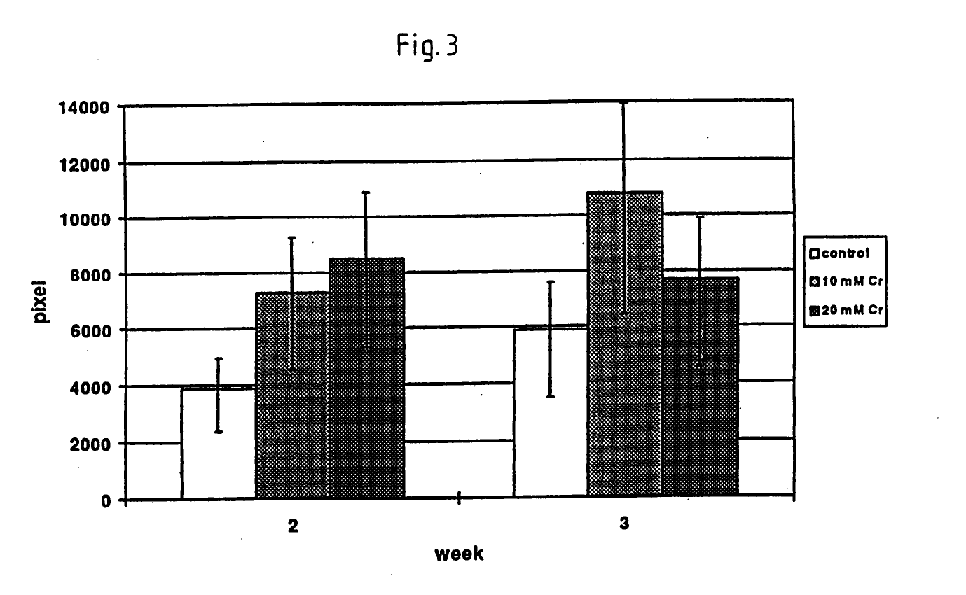

In the further experiments to quantify the calcification by image analysis of a center area (123 mm²) of the culture dish, only the creatine groups were compared to the control groups, with the GPA-treated cells not further evaluated. Statistical analysis showed that the calcified area in the 20 mM Creatine group (FIG. 3) was significantly higher than the one in the control group (p<0.02) at 2 weeks. At 3 weeks, 10 mM creatine group had more mineralization than the control, whereas the 20 mM creatine was less effective, but there was no significant difference between the various groups.

TEM-Monolayer

The ultrastructure of the control group at 1% ECS was similar to the cells kept at 10% FCS. The ultrastructure showed that there were no obvious differences between the control, the 10 mM creatine group, the 20 mM creatine group, the 1 mM GPA group, and the 5 mM GPA group.

In all groups, there was collagen production and mineralization. The cytoplasm of cells had the typical features of osteoblasts, such as a well developed rER, Golgi area, mitochondria, vesicles, micro-filaments. The cells had many cell processes that were in close contact to each other. There was abundant collagen production. The collagen fibrils were seen in membrane folds. The diameter of the fibrils was rather uniform. In the area of mineralization, the individual fibrils seem to coalesce into larger units. The mineralization pattern was similar in all groups. There were high density needle-like structures at the lowest cell layers. At the mineralization front, the same material was observed around collagen fibrils and in close opposition to the plasma membranes. Mineralized patches were seen in the collagenous matrix. In areas with high calcification, the details of the matrix were no longer visible.

Micromass Cell Culture

The NR uptake was similar in all groups at 1 and 2 weeks. At 3 weeks, the 20 mM creatine groups had a significantly lower NR uptake (p<0.005, respectively p<0.003) than the control and the 10 mM Creatine group.

The mitochondrial activity (MTT conversion) was similar in all groups at 1, 2, and 3 weeks. The creatine groups at 10 mM and 20 mM concentration, however, had a significantly higher MTT reaction at 2 weeks than at 1 week (p<0.02, respectively p<0.006). At 3 weeks, the 20 mM creatine had a significantly lower MTT conversion than at 2 weeks (p<0.015).

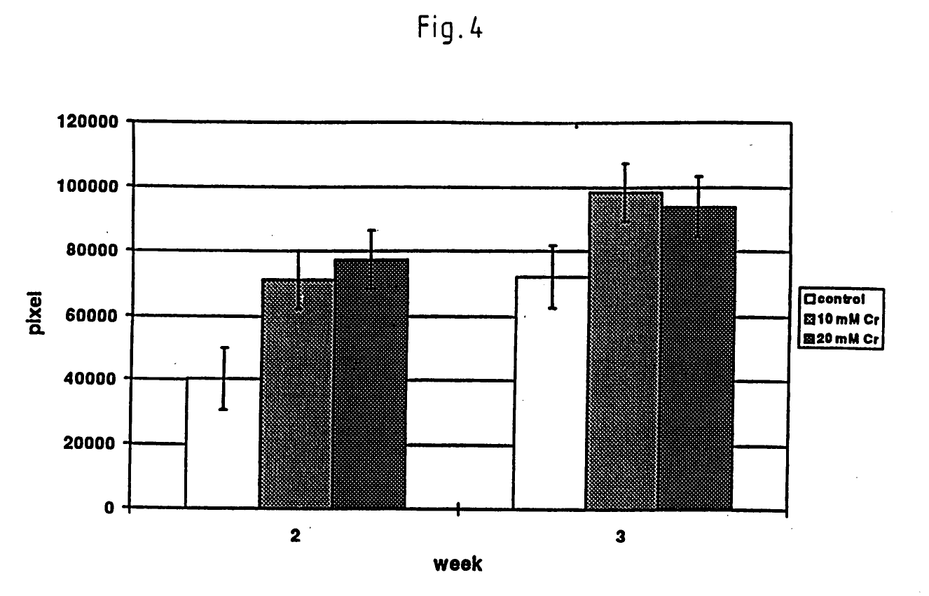

Concerning the mineralized area (FIG. 4), the creatine groups at 10 mM and 20 mM concentrations had significantly more mineralization (p<0.00025) than the control at 2 weeks. At 3 weeks, the mineralized area was significantly higher in the creatine groups at 10 mM and 20 mM concentrations than in the control (p<0.0035, respectively p<0.03). Furthermore, the control and the 10 mM creatine groups showed a significantly higher mineralization at 3 weeks than at 2 weeks (p<0.0005, respectively p<0.0015).

Organ Culture

Femora

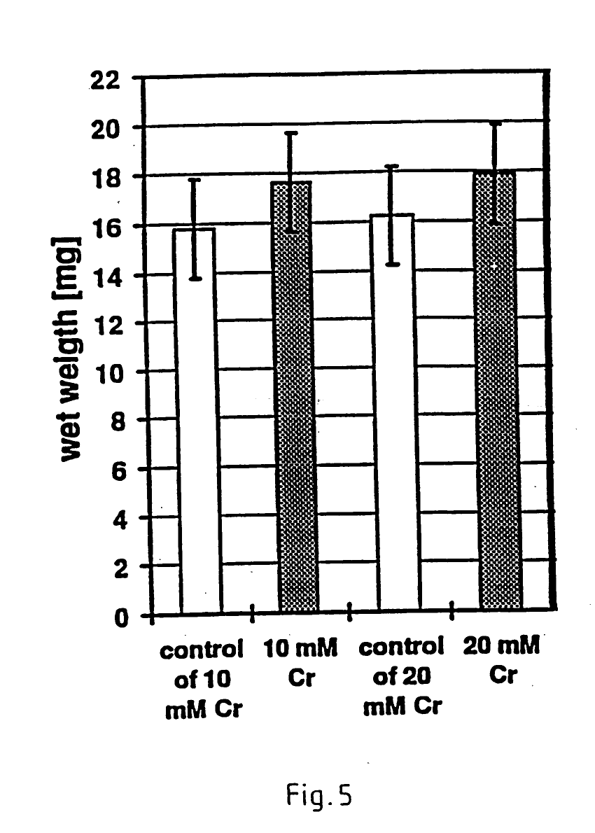

The control (FIG. 5) had significantly lower wet weights than 10 mM creatine (p<0.0005), 20 mM creatine (p<0.001), 5 mM GPA (p<0.0005) and 10 mM GPA (p<0.015). The results of 1 mM GPA were not significantly different from the control.

There was a small, but significant interaction effect of creatine in the NR uptake in monolayer cultures. This meant that the effect of the treatment with creatine was not similar at the different time points. In the control group, there was a significant increase in the NR uptake during the culture. This was due to an increase in the cell number. At 1 and 2 weeks, there was no significant difference between the groups. The effect of the 10 mM creatine on the NR uptake, however, was significant at 3 weeks-compared to that at 1 week. At 3 weeks, the 20 mM creatine group was significantly lower than the control group. This indicated some toxic effects, that resulted in a reduced proliferation of the cells. This was not observed in the 10 mM creatine group, which was similar to the control group. This showed that there was no toxic effect of the creatine at this particular concentration of 10 mM. In the microinass cultures, the NR uptake was similar in all groups at 1 and 2 weeks. At 3 weeks, however, the 20 mM creatine had significantly less than the control and the 10 mM creatine. In contrast to the monolayer cultures, the NR uptake was not reduced during culture. This could be explained by the fact that in microinass cultures, the cells were migrating off the initially inoculated drop of cells and so the cell number is slowly increasing.

The results concerning the metabolic activity of monolayer culture osteoblasts showed a significant stimulation of these cells by creatine at both concentrations, 10 mM and 20 mM, from the second week on. In the micromass cultures, the increase in the MTT conversion was only significant in the creatine groups at 2 weeks compared to the one 1 week. This indicated that the micromass cultures behave differently than the monolayer cultures. This was not astonishing, because in the micromass cultures, the cells have a very early cell-cell contact and so the differentiation process started earlier than in the monolayer cultures where the cells have first to proliferate to make cell-cell contacts. Nevertheless, the creatine significantly stimulated the metabolic activity of the micromass cultures at the early mineralization at 2 weeks, compared to 1 week.

In all groups, NR stained mainly the cells at the edge and the top of the nodules and between them. Staining with trypan blue in all groups showed that the cells at the bottom of the culture dish stained as well as those in the nodules. This could either be attributed to an artifact of staining, or it might be that the cell membrane of the stained cells was really damaged. Concerning the artifact possibility, trypan blue would also stain extracellular proteins. An indication of the presence of damaged cell membranes was obtained from the TEM ultrastructure studies of monolayer cultures. Some of the cells near the culture dish surface had electron dense, needle-like material in the cytoplasm. It could be that the lower cells of the mineralizing nodule did not get enough nutrition or oxygen by diffusion through all of the other cell layers. It is very important that the cells stay alive, because only viable cells can regulate mineral deposition and prevent dystrophic calcification. The presence of dead cells can lead to an increased mineralization.

After 2 weeks, all groups formed some small mineralized nodules that increased in size and number after 3 weeks. Calcification was also observed in single cells. Mean values were higher in the creatine groups than the control after 2 and 3 weeks. In the micromass cultures, the creatine groups had significantly more mineralization than the controls.

Thus, creatine enhanced the formation of mineralized nodules by increasing the metabolic activity of the osteoblasts in cultures. It is suggested that there is an elevation in PCr turnover during tissue mineralization, because the creatine phosphate concentration in calcified cartilage is low and the activity of the kinase in this zone is high. Furthermore, the energy metabolism in cartilage may affect the morphogenic events of skeletal growth.

There is evidence that mineralizing cells require a large amount of energy. Differentiating osteogenic cells have mitochondria with condensed cristae that represent an increased rate of energy metabolism. These mitochondria are particularly abundant in the differentiation stage and decline as the culture matures. Mineralization is thought to be associated with an optimal level of energy metabolism rather than extreme hypo- or hyperoxia.

Increased glycolysis with constant mitochondrial activity results in an augmented energy metabolism and increased ATP production. This increased availability of ATP could be a reason why osteoblasts synthesize more collagen when they are exposed to a high pH. An increased cell differentiation, during the formation of bone and cartilage, is accompanied by enhanced activities of ATPase and lactate, malate, and glucose-6-phosphate dehydrogenases. Maximum activity is observed at the onset of the matrix deposition, followed by a decrease of enzyme activities during the transformation of osteoblasts to mature osteocytes and at the hypertrophy of chondrocytes. Histochemical ATPase activity, detected in osteoblasts, parallels the metabolic activity and viability of these cells. The ATPase activity in bone and cartilage cells is far less than in skeletal muscle, blood vessels, and bone marrow. Osteoclasts reveal strong ATPase activity followed in intensity by osteoblasts, osteochondrogenic cells, and lastly, osteocytes. Cartilage cell activity, determined in this way, is generally weaker than osteoblastic activity. Young cell compartments reveal greater activity than those of older animals, with peak activity usually observed to 5 weeks of age. With increasing age and reduced functional demands, the ATPase activity diminishes except in articular cartilage cells.

Inhibition of the glycolysis causes both a reduction in collagen synthesis and a hypermineralization in tibiae of chick embryos over a wide range of [Ca×Pi] in the medium (Pi 0.5 mM to 3.0 mM and 1.8 mM Ca²⁺). Furthermore, in the absence of glutamine, there is more cell necrosis. Glutamine enters the citric acid cycle at a-ketoglutarate and provides biosynthetic precursors and NADH. NADH enters the oxidative phosphorylation and provides ATP. Inhibition of the activity of NAD-dependent enzymes associated with the production of ATP impairs cartilage formation, resulting in limb shortening.

GPA, a competitive inhibitor of creatine entry into cells, seems to have adverse effects on both the metabolism and the viability of the cells, but mineralization is increased. This could be explained by the fact that cell death can also lead to mineralization. Since metabolic activity of creatine-treated cells was generally higher compared to controls, and the same parameter was lower in GPA, it was concluded that increased mineralization in the creatine treated groups was due to the metabolic stimulation of osteoblasts, whereas the one in GPA-treated cells was mainly due to cell death. It is shown that growth plate cartilage cannot adapt to the metabolic stress imposed by GPA administration resulting in a disturbed enchondral bone formation in vivo and in vitro. The zone of calcified cartilage is wider and extends into the metaphysis. The hypertrophic chondrocytes are vacuolated and poorly columnated, and mineralization is less abundant and also occurs in the transverse septa. Vascular invasion of the tissue is poor. There is a reduction in the osteoid formation. GPA interferes with the synthesis of pro-a type II and type X collagen in cultured chondrocytes. In long-term, shell-less culture in the presence of GPA, the total CK activity is not altered, but the CK isoenzyme profile is disturbed. The activity of BB-CK is suppressed in the long bones, but the isoenzyme distribution of calvariae is not affected. Normal embryonic cartilage contains nearly equal proportions of MM-CK and BB-CK. Embryonic calvariae and bone mainly express BB-CK. Feeding of rat and mice with GPA progressively decreases the concentrations of creatine and PCr in heart and skeletal muscle, and leads to marked morphological changes mainly affecting mitochondria. A population of enlarged, rod-shaped mitochondria with characteristic crystalline intramitochondrial inclusions appears in adult rat cardiomyocytes in vitro. This phenomenon is fully reversible if the cell culture medium is supplemented with creatine. The appearance of highly ordered intra-mitochondrial inclusions correlates with a low intracellular total creatine content. Immunofluorescence and immuno-electron microscopy show that these inclusions are enriched for Mi-CK. In the GPA-treated osteoblasts, the mitochondria were similar to the control and creatine groups. Osteoblasts respond differently to GPA than do muscle cells. It is shown that GPA had comparably less influence on the creatine and PCr contents of brain. Soleus mitochondria show a four-fold increase in Mi-CK protein and a three-fold increase in adenine nucleotide translocator protein compared to the control.

Creatine stimulates, via the action of creatine kinase and other enzymes regulated by creatine or phosphocreatine, like AMP-dependent protein kinase, the mineralization of osteoblasts in culture by increasing the metabolic activity of the cells in monolayer culture. In micromass cultures, the creatine enhanced the mineralization, but the metabolic activity was similar to the control. At 2 weeks, however, the MTT conversion was significantly increased in the creatine group compared to 1 week. Creatine is believed to have some effects on the differentiation process of the cells in this cell culture model. During nodule formation and subsequent calcification, the cells need a large amount of chemical energy. Biosynthesis of matrix collagen and proteoglycans, and the proliferation of the cells are increased. Creatine, as an external energy supply, has the advantage that it does not decrease the pH in the growth medium, and thus avoids an inhibition of glycolysis and collagen synthesis.

Creatine also increases the wet weight of embryonic femora (FIG. 5) in organ culture, indicating that not only bone but also cartilage cells benefit from external creatine supply. The biosynthesis of the matrix collagen and proteoglycan, and the proliferation of the cells are stimulated.

Creatine can, therefore, be applied as a food additive or supplement for humans and animals to support the recovery after trauma and orthopaedic surgery of fractures and bone defects. Creatine also has potential to stimulate the metabolism of osteoblasts in patients suffering from osteoporosis. The treatment of degenerative cartilage diseases, such as arthritis, is also supported by creatine.

The treatment of large bone defects is still a demanding task for surgeons. Patients suffering from large bone defects can be treated with bone grafting from the illiac crest to the defect, or by applying callus distraction or segment transport. All these procedures are very painful for the patient, and additionally, the amount of bone graft is limited. The use of tissue engineering offers a solution to this problem. Bone-forming cells (osteoblasts, mesenchymal stem cells, periosteal cells, stromal bone marrow cells, or satellite cells of the muscle), as well as chondroblasts of healthy individuals, or from a patient himself, are cultured as monolayers, micromass cultures, or in a three-dimensional, biodegradable scaffold in the presence of creatine. At a later point in time, the bone or cartilage cells or cell-seeded sponges, foams, or membranes will be transferred to the defect in the patient. The most critical step in this approach is the cell culture work. It is fundamental that the cells survive, proliferate, and differentiate in vitro. Therefore, culture conditions need to be optimal. In this respect, addition of creatine to the culture medium as a supplement is beneficial.

Although bone and cartilage cells express creatine kinase, albeit at relatively low levels compared to muscle and brain cells, it is surprising that over-expression of creatine kinase together with creatine supplementation improved proliferation, metabolic stability, and resistance towards different stressors, e.g., toxins, heat, metabolic overload of cartilage and bone cells. Thus, bone forming cells (osteoblasts, periostal cells, stromal bone marrow cells, or satellite cells of muscle) and cartilage forming cells (chondroblasts) removed from healthy individuals, or from a patient to be treated, are brought into cell culture and transfected with complementary DNA coding for creatine kinase isoforms (either cytosolic muscle-type MM-CK, cytosolic ubiquitous brain-type BB-CK, or the 5, or combinations thereof). Complementary DNA (cDNA) can be obtained by reverse transcribing (RT) mRNA of CK isoenzymes, by RT-polymerase-chain reaction (RT-PCR), or by other methods using the appropriate primers corresponding to the respective CK isoenzymes.

The methods of gene transfer for cDNA's encoding for creatine kinase isoforms will encompass the entire selection of possible transfection techniques, as well as new techniques developed and made accessible to the public domain in the future, such as transfection via microinjection of cells, microsphere bombardment, or DNA-precipitate transfection, as well as transfection via various viruses, viral and non-viral vectors, or plasmids (single copy- and multi-copy plasmids), cosmids, or artificial chromosomes. Creatine kinase expression is made under the control of weak or strong tissue specific promotors. Built-in selection markers, e.g., resistance towards antibiotics, toxins, or others, make it possible to select for trausfected cells that are then expanded in cultures as described above in the presence of 1 to 20 mM creatine.

Cartilage or bone cells transfected with creatine kinase cDNA, made to overexpress creatine kinase isoenzyme(s), are then selected on a selection medium and expanded and cultivated either as monolayers, micromass cultures, or on three-dimensional, biodegradable scaffolds or tissue sponges (as described above) to form in vitro genetically engineered cartilage- and bone pre-tissues which can be transplanted into the areas of cartilage or bone defects. For example, such transfected cartilage cells can be injected into arthritic joints to repopulate the areas of defect and repair chondro-degenerative defects in this joint by proliferation and producing new chondrocyte-derived extracellular matrix. Similarly, transfected bone-forming cells can be reimplanted into areas of bone defect to initiate regeneration and growth of bone mass in patients.

Since creatine kinase and creatine/phosphocreatine play an important role in the generation and maintenance of cartilage-and bone tissues, such tissues, genetically engineered to overexpress creatine kinases and being supplemented by externally added creatine or creatine analogues, are growing better after transplantation into areas of cartilage or bone defect in patients supplemented orally or locally with creatine.

Genetic engineering of creatine kinase into cartilage and bone cells, in conjunction with creatine supplementation, improves the proliferation growth, and specific function of these cells, e.g., the formation of extracellular cartilage- or bone-specific matrix. This metabolic engineering procedure, followed by creatine supplementation, is beneficial for cartilage and bone formation, healing and repair, as well as for mineralization.

The concentration of the creatine compound in the culture medium should preferably be in the range of 10 to 20 mM. The culture medium typically contains 0.1% to 5.0%, preferably 0.5% to 2% foetal calf serum. Furthermore, the culture medium should contain 10 to 250 μg, preferably 25 to 100 μg, ascorbic acid or an equivalent amount of a pharmaceutically acceptable ascorbate. The cell culture is started with 2,000 to 100,000 cells, preferably 10,000 to 50,000 cells.

In a preferred embodiment of the invention, the creatine compound is administered in combination with hormones, preferably selected from parathyroid hormone-related protein, thyroid hormone, insulin, sex steroids (estrogen, androgen, testosterone), prostaglandins, and glucocorticoids.

In a further preferred embodiment, the creatine compound is administered in combination with intermittent administration of parathyroid hormone, preferably in combination with 1,25(OH)₂ vitamin D₃ and analogues or metabolites of vitamin D, calcitonine, estrogen, or bisphosphonates.

A further preferred embodiment includes administration of the creatine compound in combination with vitamins, preferably selected from 1.25(OH)₂ vitamin D₃ and analogues or metabolites of vitamin D, of vitamin C/ascorbate, and of retinoids.

A further preferred embodiment includes administration of the creatine compound in combination with growth factors, preferably selected from insulin like growth factors (IGF), transforming growth factor b family (TGF-b), bone morphogenic proteins (BMP), basic fibroblastic growth factor (bFGF), platelet derived growth factor (PDGF), and epidermal growth factor (EGF).

A further preferred embodiment includes administration of the creatine compound in combination with cytokines, preferably selected from interleukins (IL), interferons, and leukemia inhibitory factor (LIF).

A further preferred embodiment includes administration of the creatine compound in combination with matrix proteins, preferably selected from collagens, glycoproteins, hyaluronan, and proteoglycans.

Suitable glycoproteins include:
a) alkaline phosphatase,
b) osteonectin (ON),
c) gamma-carboxy glutamic acid-containing proteins, preferably matrix gla protein, or osteocalcin or bone gla protein (OC), and
d) arginine-glycine-asparagine-containing proteins, preferably thromspondin, fibronectin, vitronectin, fibrillin, osteoadherin, sialoproteins (osteopontin or bone sialoprotein BSP).

Suitable proteoglycans include:
a) aggrecan,
b) versican,
c) biglycan, and
d) decorin

In a further preferred embodiment, the creatine compound is administered in combination with serum proteins, preferably selected from albumin and alpha-2H5 glycoprotein.

A further preferred embodiment includes administration of the creatine compound in combination with enzymes, preferably selected from metalloproteinases, collagenases, gelatinases, stromelysins, plasminogen activators, cysteine proteinases, and aspartic proteinases.

A further preferred embodiment includes administration of the creatine compound in combination with calcium salts, bone meal, or hydroxyapatite.

A further preferred embodiment includes administration of the creatine compound in combination with fluoride salts, preferably sodium fluoride, or monosodium fluorophosphate.

A further preferred embodiment includes administration of the creatine compound in combination with peptides, preferably selected from amylin, vasoactive agents, and neuropeptides.

A further preferred embodiment includes administration of the creatine compound in combination with antioxidants, preferably selected from cysteine, N-acetyl-cysteine, glutathions and vitamins A, C, D, or E.

A further preferred embodiment includes administration of the creatine compound in combination with a substance selected from transferrin, selenium, boron, silicon, or nitric oxide.

In a preferred embodiment of the invention, the agent is essentially free of dihydrotriazine. It has been found that dihydrotriazine is a toxic impurity of commercially available creatine and that it has an adverse effect for the patient. For the same reason, the agent should be essentially free of dicyano-diamide, which is also a toxic impurity of commercially available creatine.

It is further advantageous to an agent that is essentially free of creatinine as a natural degradation product of creatine. The agent according to the invention is administered to a human patient preferably in an amount of 1.4 to 285 mg per day.

In a further preferred embodiment of the invention, the creatine analogue has the general formula:
Z₁-C(-Z₂)-X-A-Y
and pharmaceutically acceptable salts thereof, wherein:
Y is selected from: —CO₂H, —NI—OH, —NO₂, —SO₃H, —C(═O)NHSO₂J, and —P(═O)(OH)(OJ),
   wherein J is selected from: hydrogen, C₁-C₆ straight chain alkyl, C₃-C₆ branched alkyl, C₂-C₆ straight alkenyl, C₃-C₆ branched alkenyl and aryl;
A is selected from: C, CH, C₁-C₅ alkyl C₂-C₅ alkenyl, C₂-C₅ alkynyl, and C₁-C₅ alkoyl chain, each having 0-2 substituents which are selected independently from:
   K, where K is selected from: C₁-C₆ straight alkyl, C₂-C₆ straight alkenyl, C₁-C₆ straight alkoyl, 3-6 branched alkyl, C₃-C₆branched alkenyl, C₄-C₆ branched alkoyl, K having 0-2 substituents independently selected from: bromo, chloro, epoxy and acetoxy,
   an aryl group selected from: a 1-2 ring carbocycle and a 1-2 ring heterocycle, wherein the aryl group contains 0-2 substituents independently selected from: —CH₂L and —COCH₂L
      where L is independently selected from: bromo, chloro, epoxy and acetoxy, and
   —NH-M, wherein M is selected from: hydrogen, C₁-C₄ alkyl C₂-C₄ alkenyl, C₁-C₄alkoyl, C₃-C₄branched alkyl, C₃-C₄ branched alkenyl, and C₄-C₆branched alkoyl;
X is selected from: NR₁, CHR₁, CR₁, O and 5,
   wherein R₁ is selected from:
      hydrogen,
      K where K is selected from: C₁-C₆ straight alkyl, C₂-C₆ straight alkenyl, C₁-C₆ straight alkoyl, ₃-C₆ branched alkyl, C₃-C₆branched alkenyl, and C₄-C₆ branched alkoyl, K having 0-2 substituents independently selected from: bromo, chloro, epoxy and acetoxy,
      an aryl group selected from: a 1-2 ring carbocycle and a 1-2 ring heterocycle, wherein the aryl group contains 0-2 substituents independently selected from: —CH₂L and —COCH₂L, where L is independently selected from: bromo, chloro, epoxy and acetoxy;
      a C₅-C₉ Alpha-amino-omega-methyl-omega-adenosyl carboxylic acid attached via the omega-methyl carbon;
   a C₅-C₉ Alpha-amino-omega-aza-omega-methyl-omega -adenosylcarboxylic acid attached via the omega-methyl carbon; and
   a C₅-C₉ Alpha-amino-omega-thia-omega-methyl-omegaadenosylcarboxylic acid wherein A and X are connected by a single or double bond;
Z₁ and Z₂ are chosen independently from the group of: ═O, —NHR₂, —CH₂R₂, —NR₂OH; wherein, Z₁ and Z₂ may not both be ═O and wherein R₂ is selected from:
   hydrogen;
   K, where K is selected from: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from bromo, chloro, epoxy and acetoxy;
   an aryl group selected from: a 1-2 ring carbocycle and a 1-2 ring heterocycle, wherein the aryl group contains 0-2 substituents independently selected from —CH₂L and —COCH₂L where L is independently selected from: bromo, chloro, epoxy and acetoxy;
   a C₄-C₈ Alpha-amino-carboxylic acid attached via the omega-carbon;
   B, wherein B is selected from: —CO₂H, —NHOH, NO₂, —SO₃H, —C(═O)NHSO₂J and —P(═O)(OH)(OJ),
      wherein J is selected from: hydrogen C₁-C₆ straight alkyl, C₃-C₆ branched alkyl, C₂-C₆ straight alkenyl, C₃-C₆ branched alkenyl and aryl; wherein B is optionally connected to the nitrogen via a linker selected from: C₁-C₂ alkyl, C₂ alkenyl, and alkoyl;
   —D-E, wherein D is selected from: C₁-C₃ straight chain alkyl, C₃ branched alkyl, C₂-C₃ straight alkenyl, C₃ branched alkenyl, C₁-C₃ straight alkoyl, and aryl; and E is selected from: —(PO₃)ₙNMP, where n is 0-2 and NMP is a ribonucleotide monophosphate connected via the 5′-phosphate, 3′-phosphate or the aromatic ring of the base; —[P(═O) (OCH₃)(O)]ₘ-Q, wherein m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose of the aromatic ring of the base; and an aryl group containing 0-3 substituents chosen independently from: Cl, Br, epoxy, acetoxy, —OG, —C(═O)G, and —CO₂G, where G is independently selected from: C₁-C₆ straight alkyl, C₂-C₆ straight alkenyl, C₁-C₆ straight alkoyl, C₃-C₆ branched alkyl, C₁-C₆ branched alkenyl, C₄-C₆ branched alkoyl; wherein E may be attached at any point to D, and if D is alkyl or alkenyl, D may be connected at either or both ends by an amide linkage; and
-E, wherein E is selected from: —(PO₃)ₙNMP, where n is 0-2 and NMP is a ribonucleotide monophosphate connected via the 5′-phosphate, 3′-phosphate or the aromatic ring of the base; —P(P(═O)(OCH₃)(O))ₘ-Q wherein in is 0-3 and Q is a ribonucleoside connected via the ribose or the aromatic ring of the base; —[P(═O)(OH)(CH₂)]ₘ-Q, wherein in is 0-3 and Q is a ribonucleoside connected via the ribose of the aromatic ring of the base; and an aryl group containing 0-3 substituents chosen independently from: Cl, Br, epoxy, acetoxy, -)G. —C(═O)G, and —CO₂G, where G is independently selected from: C₁-C₆ straight alkyl, C₂-C₆ straight alkenyl, C₁-C₆ straight alkoyl; C₃-C₆branched alkyl, C₃-C₆ branched alkenyl, C₄-C₆ branched alkoyl; and if E is aryl, B may be connected by an amide linkage;
if R₁ and at least one R₂ group are present, R₁ may be connected by a single or double bond to an R₂ group to form a cycle of 5 to 7 members;
if two R₂ groups are present, they may be connected by a single or double bond to form a cycle of 5 to 7 members; and
if R₁ is present and or Z₂ is selected from —NHR₂, —CH₂R₂ and —NR₂OH, then R₁ may be connected by a single or double bond to the carbon or nitrogen of either Z₁ or to form a cycle of 4 to 7 members.

The various modifications and preferred embodiments characterized in the dependent claims have produced a stimulatory effect on bone or cartilage. While the foregoing description and drawings represent the preferred embodiments of the present invention, it will be obvious for those of ordinary skill in the art that various changes and modifications may be made therein, without departing from the true spirit and scope of the present invention.

1. A method of treating at least one bone or cartilage condition which comprises administering to an animal a therapeutically effective amount of an agent comprising creatine, or an analogue or pharmaceutically acceptable salt thereof, to treat bone or cartilage conditions.

2. The method of claim 1, wherein the animal is a mammal and the condition comprises a bone or cartilage disease, a bone fracture or defect, or a degenerative disease of cartilage.

3. The method of claim 2, wherein the mammal is a human and the disease comprises osteoporosis, osteoarthritis, or periodontitis.

4. The method of claim 2, wherein the mammal is a human and the agent is incorporated in a bone or cartilage graft that is applied to the bone fracture or defect.

5. The method of claim 4, wherein the agent is incorporated in at least one three dimensional construct of osteoblasts, chondrocytes, or mesenchymal stem cells designed for tissue engineering of the bone or cartilage condition and wherein the construct is administered to the bone or cartilage.

6. The method of claim 5, further comprising:
obtaining bone or cartilage forming cells from a healthy individual;
culturing the bone or cartilage forming cells in the presence of the agent to form a three-dimensional cell assembly; and
transferring the three-dimensional cell assembly to a specific location having a bone or cartilage defect of the patient.

7. The method of claim 1, wherein the creatine, or analogue or pharmaceutically acceptable salt thereof, comprises creatine, creatine phosphate, creatine pyruvate, cyclocreatine, homocreatine, or homocyclocreatine.

8. The method of claim 1, further comprising administering at least one of: hormones, vitamins, growth factors, cytokines, matrix proteins, serum proteins, enzymes, calcium salts, fluoride salts, bone meal, hydorxyapatite, peptides, antioxidants, transferrin, selenium, boron, silicon, or nitric oxide.

9. The method of claim 8, wherein, when administered, the hormones comprise parathyroid hormone-related protein, thyroid hormone, insulin, a sex steroid, prostaglandins, or glucocorticoids; the vitamins comprise 1,25(OH)₂ vitamin D₃ and analogues or metabolites of vitamin D, vitamin C/ascorbate, or retinoids; the growth factors comprise insulin-like growth factors (IGF), transforming growth factor b family (TGF-b), bone morphogenic proteins (BMP), basic fibroblastic growth factor (bFGF), platelet derived growth factor (PDGF), or epidermal growth factor (EGF); the cytokines comprise interleukins (IL), interferons, or leukaemia inhibitory factor (LIF); the matrix proteins comprise collagens, glycoproteins, hyaluronan, or proteoglycans; the serum proteins comprise albumin or alpha-2H5 glycoprotein; the enzymes comprise metalloproteinases, collagenases, gelatinases, stromelysins, plasminogen activators, cysteine proteinases, or aspartic proteinases; the fluoride salts comprise sodium fluoride or monosodium fluorophosphate; the peptides comprise amylin, vasoactive agents, or neuropeptides; the antioxidants comprise cysteine, N-acetyl-cysteine, glutathions, or vitamins A, C, D, or E.

10. The method of claim 9, wherein, when matrix proteins are administered, the matrix proteins are glycoproteins comprising alkaline phosphatase, osteonectin (ON), gamma-carboxy glutamic acid-containing proteins, or arginine-glycine-asparagine-containing proteins, or proteoglycans comprising aggrecan, versican, biglycan, or decorin.

11. The method of claim 9, wherein the hormone is parathyroid hormone and the parathyroid hormone is administered intermittently with the agent.

12. The method of claim 11, further comprising administering 1,25(OH)₂ vitamin D₃ and analogues or metabolites of vitamin D, calcitonine, estrogen, or bisphosphonates to the individual.

13. The method of claim 1, wherein the bone comprises cells comprising osteoblasts, periosteal cell, stromal bone marrow cells, satellite cells of muscle tissue, or mesenchymal stem cells, or a combination thereof.

14. The method of claim 1, wherein the cartilage comprises cells comprising chondroblasts or mesenchymal stem cells.

15. The method of claim 5, wherein the stem cells are cultured as monolayers, micromass cultures, or in a three-dimensional biodegradable scaffold.

16. The method of claim 6, wherein the three-dimensional cell assembly has a structure of a seeded sponge, foam, or membrane.

17. The method of claim 6, wherein 10 to 20 mM of creatine is concentrated in a culture medium containing one of 0.1% to 5% fetal calf serum or 10 to 250 μg of ascorbic acid or an equivalent amount of a pharmaceutically acceptable ascobate.

18. The method of claim 6, wherein a cell culture is started with 2,000 to 100,000 cells.

19. The method of claim 1, wherein the agent is essentially free of one or more of dihydrotriazine; dicyano-diamide; or creatinine.

20. The method of claim 1, wherein the agent is administered to a human patient in an amount of 1.4 to 285 mg per day.

21. The method of claim 1, wherein the creatine analogue has the general formula:
Z₁-C(-Z₂)-X-A-Y
and pharmaceutically acceptable salts thereof, wherein:
Y is selected from: —CO₂H, —NI—OH, —NO₂, —SO₃H, —C(═O)NHSO₂J, and —P(═O)(OH)(OJ), wherein J is selected from: hydrogen, C₁-C₆ straight chain alkyl, C₃-C₆ branched alkyl, C₂-C₆ straight alkenyl, C₃-C₆ branched alkenyl and aryl;
A is selected from: C, CH, C₁-C₅ alkyl, C₂-C₅ alkenyl, C₂-C₅ alkynyl, and C₁-C₅ alkoyl chain, each having 0-2 substituents which are selected independently from:
K, where K is selected from: C₁-C₆straight alkyl, C₂-C₆ straight alkenyl, C₁-C₆ straight alkoyl, 3-6 branched alkyl, C₃-C₆ branched alkenyl, C₄-C₆ branched alkoyl, K having 0-2 substituents independently selected from: bromo, chloro, epoxy and acetoxy; an aryl group selected from: a 1-2 ring carbocycle and a 1-2 ring heterocycle, wherein the aryl group contains 0-2 substituents independently selected from: —CH₂L and —COCH₂L, wherein L is independently selected from: bromo, chloro, epoxy and acetoxy; and
—NH-M, wherein M is selected from: hydrogen, C₁-C₄ alkyl, C₂-C₄ alkenyl, C₁-C₄ alkoyl, C₃-C₄ branched alkyl, C₃-C₄ branched alkenyl, and C₄-C₆ branched alkoyl;
X is selected from: NR₁, CHR₁, CR₁, O and 5,
wherein R₁ is selected from:
hydrogen,
K where K is defined above; and
an aryl group selected from: a 1-2 ring carbocycle and a 1-2 ring heterocycle, wherein the aryl group contains 0-2 substituents independently selected from: —CH₂L and —COCH₂L where L is defined above;
a C₅-C₉ Alpha-amino-omega-methyl-omega-adenosyl carboxylic acid attached via the omega-methyl carbon;
a C₅-C₉ Alpha-amino-omega-aza-omega-methyl-omega -adenosylcarboxylic acid attached via the omega-methyl carbon; and
a C₅-C₉ Alpha-amino-omega-thia-omega-methyl-omegaadenosylcarboxylic acid wherein A and X are connected by a single or double bond;
Z₁ and Z₂ are chosen independently from: ═O, —NHR₂, —CH₂R₂, —NR₂OH; wherein, Z₁ and Z₂ may not both be ═O and wherein R₂ is selected from:
hydrogen;
K, where K is defined above;
an aryl group selected from: a 1-2 ring carbocycle and a 1-2 ring heterocycle, wherein the aryl group contains 0-2 substituents independently selected from: —CH₂L and —COCH₂L where L is as defined above;
a C₄-C₈ Alpha-amino-carboxylic acid attached via the omega-carbon;
B, wherein B is selected from: —CO₂H, —NHOH, No₂, —SO₃H, —C(═O)NHSO₂J and —P(═O)(OH)(OJ), wherein J is as defined above:
D-E, wherein D is selected from: C₁-C₃ straight chain alkyl, C₃ branched alkyl, C₂-C₃ straight alkenyl, C₃ branched alkenyl, C₁-C₃ straight alkoyl, and aryl; and E is selected from: —(PO₃)ₙNMP, where n is 0-2 and NMP is a ribonucleotide monophosphate connected via the 5′-phosphate, 3′-phosphate or the aromatic ring of the base; —[P(=0) (OCH₃)(O)]ₘ-Q, wherein m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose of the aromatic ring of the base; and an aryl group containing 0-3 substituents chosen independently from: Cl, Br, epoxy, acetoxy, —OG, —C(═O)G, and —CO₂G, where G is independently selected from: C₁-C₆ straight alkyl, C₂-C₆ straight alkenyl, C₁-C₆straight alkoyl, C₃-C₆ branched alkyl, C₁-C₆branched alkenyl, C₄-C₆branched alkoyl; wherein E may be attached at any point to D, and if D is alkyl or alkenyl, D may be connected at either or both ends by an amide linkage; and
E, wherein E is as defined above, provided that:
when E is aryl, B may be connected by an amide linkage;
if R₁ and at least one R₂ group are present, R₁ may be connected by a single or double bond to an R₂ group to form a cycle of 5 to 7 members;
if two R₂ groups are present, they may be connected by a single or double bond to form a cycle of 5 to 7 members; and
if R₁ is present and or Z₂ is selected from —NHR₂, —CH₂R₂ and -NR2OH, then R₁ may be connected by a single or double bond to the carbon or nitrogen of either Z₁ or to form a cycle of 4 to 7 members.

22. A method of promoting growth and mineralization of bone or cartilage cells and tissues which comprises administering to a subject in need of such treatment a therapeutically effective amount of an agent comprising creatine, or an analogue or pharmaceutically acceptable salt thereof, to promote growth and mineralization of bone or cartilage therein.

23. A method of improving acceptance and osseous integration of bone implants which comprises administering to a subject in need of such treatment a therapeutically effective amount of an agent comprising creatine, or an analogue or pharmaceutically acceptable salt thereof, to improve acceptance and osseous integration of bone implants.

24. A method for accelerating healing in a subject having a defect in bone or cartilage tissue caused by trauma, surgery, or a degenerative disease, which method comprises administering to the subject a therapeutically effective amount of a creatine compound, analogue or pharmaceutically acceptable salt thereof, or a creatine kinase.

25. A composition useful for the treatment of-detects in none or cartilage tissue of animals or humans caused by trauma or surgery, the composition comprising a creatine compound, analogue or pharmaceutically acceptable salt thereof, the composition being suitable for oral administration and including a pharmacologically suitable carrier to improve bioavailability.

26. The composition of claim 25, wherein the carrier is selected from: carbohydrates, maltodextrins, and dextrose.

27. A method of preparing an agent for treatment of bone or cartilage cells or tissues, comprising:
removing bone or cartilage forming cells from a healthy subject;
adding the bone or cartilage forming cells to a cell culture;
transfecting the bone or cartilage forming cells with complementary DNA coding for creatine kinase isoforms and made to overexpress creatine kinase isoenzyme(s); and
expanding and cultivating the bone or cartilage forming cells to form in vitro genetically engineered cartilage or bone tissues transplantable into areas of cartilage or bone defects of the healthy subject.
